(19) United States
(12) Patent Application Publication
(10) Pub. No.: US 2018/0028074 A1
(43) Pub. Date: Feb. 1, 2018

(54) DEVICE FOR MONITORING A STATE OF A LIVING BEING AND CORRESPONDING METHOD

(71) Applicant: Fraunhofer-Gesellschaft zur Foerderung der angewandten Forschung e.V., Munich (DE)

(72) Inventors: Thomas BERTSCH, Schwetzingen (DE); Bernd LANGENSTEIN, Nuernberg (DE); Michael JANK, Erlangen (DE); Susanne OERTEL, Pettstadt (DE); Nadine Ramona LANG, Erlangen (DE); Matthias STRUCK, Fuerth (DE); Esther Ann RENNER, Erlangen (DE); Christian HOFMANN, Nuernberg (DE)

(21) Appl. No.: 15/660,878

(22) Filed: Jul. 26, 2017

(30) Foreign Application Priority Data
Jul. 28, 2016 (EP) 16181681.4

(51) Int. Cl. A61B 5/0205; A61B 5/00; A61B 10/00; A61F 13/537; A61B 5/145; A61B 5/1486

(52) U.S. Cl. CPC A61B 5/0205; A61B 5/14517; A61B 5/1486; A61B 10/0064; A61F 13/53708; A61B 5/0002; A61B 5/6831; A61B 5/08

(57) ABSTRACT

A device for monitoring a state of a living being includes a first sensor, a second sensor and an evaluation device. The first sensor non-invasively generates a measurement value based on a content of ammonia in the sweat of the living being. The second sensor generates a measurement value with respect to the respiration or the cardiac activity of the living being. The evaluation device generates a scalar measure of a stress of the living being based on the measurement values of the first and second sensors. Furthermore, the invention relates to a corresponding method.

### CROSS-REFERENCE TO RELATED APPLICATIONS

This application claims priority from European Patent Application No. 16181681.4, which was filed on Jul. 28, 2016, and is incorporated herein in its entirety by reference.

The invention relates to a device for monitoring a state of a living being. For example, the state includes several vital values of the living being or, e.g., a stress state. In this case, the living being is, e.g., a human being or an animal. Furthermore, the invention relates to a method for monitoring a state of a living being.

### BACKGROUND OF THE INVENTION

In the conventional technology, there are several wearable computers or wearable sensors which may be worn by a user directly at the body, e.g., as a piece of clothing. For example, such “wearables” allow for monitoring the heart of a human being. It is also known to use biosensors which, e.g., determine the lactate value in the sweat of a human being (e.g., see [1]). In this case, such lactate measurements are based on the performance diagnostics in sports-medical examinations. In this case, the lactate value is determined invasively, for which the athlete has to take a break.

In the conventional technology, approaches for non-invasive measuring can be found. One approach is to analyze sweat at the wrist using a type of bracelet, e.g., see [2]. Furthermore, it is known to insert electrodes of sensors (e.g., WO 2011/156095 A2) or components of electronic elements (e.g., US 2014/0024915 A1) into textiles.

### SUMMARY

According to an embodiment, a device for monitoring a state of a living being may have several first sensors, a second sensor and an evaluation device, wherein a first sensor is configured in such a way as to non-invasively generate a measurement value based on a content of ammonia in the sweat of the living being, and a further first sensor is embodied identically, or a further first sensor is configured in such a way as to generate a measurement value for a content of a component in the sweat of the living being other than ammonia, wherein the first sensors may be positioned at different positions with different muscle groups of the living being, wherein the first sensors each have an ion-selective electrode printed on a film, an ion-selective membrane, an ion-selective material in the form of an ionophore, a reference electrode and a counter electrode, wherein the second sensor is configured in such a way as to generate a measurement value with respect to the respiration or the cardiac activity of the living being, wherein the first sensors and the second sensor are arranged in a carrier device wearable at a body of the living being, and wherein the evaluation device is configured in such a way as to generate a scalar measure of a stress of the living being based on the measurement values of the first sensors and the measurement value of the second sensor.

The invention solves the object by a device for monitoring a state of a living being. The device comprises at least one first sensor, a second sensor and an evaluation device. The first sensor is configured in such a way as to non-invasively generate a measurement value based on a content of ammonia in the sweat of the living being. The second sensor is configured in such a way as to generate a measurement value with respect to the respiration or the cardiac activity of the living being. Finally, the evaluation device is configured in such a way as to generate a scalar measure of a stress of the living being based on the measurement value of the first sensor and the measurement value of the second sensor. In this case, the scalar measure results from the at least two measurement values and, in a configuration, is a combination of values or just a single value and, in a further configuration, is a type of general statement about a stress state of the living being to be examined. In this way, in a configuration, the scalar measure allows for a statement as to if the stress is to be maintained or reduced. Practically, in an application, this means that the living being should decrease an (e.g., athletic) activity or is able to maintain the same with respect to the stress.

All in all, at least two measurement values of two different measured quantities are combined into a scalar measure. In this case, this measure describes the stress of the living being as a state of the living being.

In a configuration, in order to determine the measure, data sets are used in which the measurement results determined non-invasively by the device are matched with measurement values determined invasively. In this way, e.g., in the measurements for the data sets, the lactate value is determined invasively from the blood as a measure of the stress. Advantageously, this is done in a sufficiently large number of subjects in order to obtain data sets which allow for mapping at least two values (e.g., ammonia content and respiration or ammonia content and heart beat) to the scalar measure of the stress of the living being. Thus, the data sets consist of, e.g., tables, algorithms and/or formulas.

In particular, the device allows for a quick, continuous, non-invasive, i.e., in particular bloodless, vital parameter measurement which, in a further configuration, is extended to a state assessment. In this case, a direct data evaluation of measurement values advantageously occurs.

In the device according to the invention, ammonia (chemical name: NH₃) is measured via the first sensor. Ammonia is generated within the Purine Nucleotide Cycle. In performance diagnostics, the ammonia content in the blood was already examined and recognized as being significant [8].

This is an advantage over the lactate measurement for which it has been unambiguously proven in the last few years that the performance diagnostics using the lactate value are only significant by a blood examination, i.e., by an invasive procedure. It has been verified several times that the concentration of the lactate in the sweat does not correlate with the concentration in the blood. Rather, the sweat glands themselves produce lactate and, thus, render an unambiguous diagnostic using the sweat impossible. In contrast, it could be proven hat ammonia in the sweat is mainly washed out of the blood [3-6]. It can be concluded from tests that a physiological connection between a blood value and a sweat value is given.

The device according to the invention allows for monitoring a living being, wherein the device—in a configuration—may be worn at the body and, besides the actual measurement, also carries out a direct evaluation and, advantageously with respect to reference values, also an assessment of the measurement values.

On the one hand, the measurements are made with regard to the ammonia content in the sweat and, on the other hand, at least to the respiration or the cardiac activity. In this case, in a configuration, the respiration and cardiac activity are measured by a corresponding number of second sensors. Thus, in a configuration, several second sensors are present, wherein a second sensor generates a measurement value with respect to the respiration, and another second sensor generates a measurement value with respect to the cardiac activity. Additionally, further measurement values are generated which relate to, e.g., other components of the sweat.

For example, JP 2006-43120 A2 or US 2014/0012114 A1 show a measurement arrangement for the measurement of sweat. For example, the measurement of ammonia in liquids is known from U.S. Pat. No. 4,700,709 A.

Thus, in a configuration, the evaluation device correlates the measurement value of the first sensor with the measurement value of the at least one second sensor which relates to established vital parameters such as the ECG (heartrate and heartrate variability) and/or the respiration rate.

In particular, in a configuration, all measurement values are determined non-invasively by the device. This simplifies the usage of the device and also prevents possible complications for the living being.

In a configuration, the first sensor and the second sensor are arranged in a carrier device wearable at a body of the living being. For example, the carrier device is a piece of textile clothing or a type of watch, or bracelet, or another type of strap, which may be worn around a body part.

In a configuration, the evaluation device is arranged separately from the sensors and the carrier device, and is connected to the first and the second sensor, e.g., by radio.

In a further configuration, the first sensor, the second sensor and the evaluation device are arranged in such a carrier device wearable at a body of the living being. Therefore, in this configuration, the device is located entirely in a carrier device in order to be worn by the living being at the body.

In a configuration, the evaluation device is configured in such a way as to offset movement artefacts.

In an application, the state of the living being to be monitored is, in particular, stress states, e.g., during athletic activity or during medical treatment.

In a configuration, the device comprises several first sensors. In a configuration, the first sensors are located at different positions so that the sweat may be analyzed at different locations of the living being. In this way, e.g., different muscle groups may be specifically examined. Furthermore, in a configuration, the first sensors are arranged such that movement artefacts in the measurement generally offset themselves. In other configurations, redundancy is provided by the several first sensors so that internal matching of the first sensors is also made possible. In a configuration, the first sensors are embodied in the same way. In an alternative configuration, at least two first sensors differ from one another. In a configuration, an additional sensor is present which generates a measurement value for a content of a component (e.g., lactate or an electrolyte) in the sweat other than ammonia.

In a configuration, the device comprises at least one transport device. In this case, the transport device is configured in such a way as to transport the sweat to the first sensor and/or away from the first sensor. In a configuration, a transport device allows for placing the first sensor offset from the direct contact area to the living being. Alternatively or additionally, an essentially continuous flow of sweat as the measurement medium is enabled by the transport device and, thus, current measurements are ensured.

In a configuration, the transport device comprises at least one component provided with at least one channel. Thus, the component may also be understood as a guiding component which guides the sweat to the first sensor and/or away from the same in, e.g., micro-structured channels or capillaries.

In a configuration, the transport device comprises at least one component comprising an absorbent material. In a configuration, the component comprises materials having different absorbencies.

In a configuration, the transport device comprises at least one pumping device. In this case, such a pump is advantageously a micro pump which appropriately moves the sweat.

In a configuration, the first sensor comprises at least one ion-selective electrode printed on a film.

An ion-selective electrode (other terms are: ion-specific electrode or ion-sensitive electrode) allows for measuring the concentration and/or the activity of a specific solved ion. For this, the ion-selective electrode and a second electrode—the reference electrode—are introduced into a measurement solution, and the voltage between the electrodes is measured.

In this case, in a configuration, the ion-selective electrode is, in particular, configured in such a way as to measure ions which allow for determining the ammonia. Therefore, in a configuration, the ion-selective electrode allows for measuring the cation ammonium (chemical name: NH₄⁺).

Based on the fact that different ions comprising similar characteristics are present in the sweat, in a configuration, the evaluation device is embodied in such a way as to consider, during evaluation of the measurement values of the first sensor, the presence of different ions.

In this case, the application onto a—in particular—flexible film helps the accommodation in said carrier device and does not hinder the wearing comfort.

In a configuration, the film is transparent.

In a further configuration, the film at least partially consists of polyester (PET or PEN) or of polyimides (PI) or of other synthetic materials such as polyurethane (PU) or of synthetic textiles.

In a configuration, the first sensor comprises an ion-selective membrane, a reference electrode and a counter electrode. In this case, the membrane allows, in particular, the ions to be measured to pass.

In a configuration, the membrane is embodied in such a way as to allow ammonium ions (NH₄⁺) to pass. In a configuration, since such a membrane also allows to pass, e.g., cations such as K⁺ or Na⁺, corresponding calibration data is available to the evaluation device in order to offset such an adverse effect on the measurement values.

All in all, the first sensor is advantageously configured in a small, flexible, wearable, skin-compatible, textile-integrable, cost-effective and energy-efficient manner.

Advantageously, it is a printed first sensor comprising printed operating electrodes and reference electrodes as well as at least one passivation layer.

Advantageously, manufacturing is carried out with commercially available screen-printing pastes, wherein, in a configuration, an optimization of the printed electrodes is carried out by means of baking at paste-specific temperatures. In a configuration, the preparation of the reference electrode includes applying a mixture of polyvinyl butyral, methanol and sodium chloride (cf. [9]).

In a configuration, manufacturing the first sensor includes applying the ion-selective material in the form of an ionophore (such as nonactin, valinomycin, sodium ionophore) onto an operating electrode. In this case, in a configuration, a mixture with a matrix (network-forming materials, in particular polymers and advantageously polyvinyl butyral, PVB, and polyvinyl chloride, PVC) is provided.

In a configuration, the amount of the ionophore involved is reduced as far as possible such as to realize a cost-effective sensor which may also be understood as disposable product.

In a configuration, the device comprises an output device. In this case, the output device is configured in such a way as to output the scalar measure generated by the evaluation device. In a configuration, the output device transmits the measure to a display unit, e.g., which may be worn by an athlete in the form of a watch. Alternatively or additionally, the measure is transmitted to a computer or a similar processing unit.

In a configuration, the output device is configured in such a way as to output the measure by radio.

In a configuration, the device comprises a data memory. In this case, the evaluation device is configured in such a way as to generate the scalar measure based on the measurement value of the first sensor and at least the measurement value of the second sensor, as well as based on the data stored in the data memory. The data in the data memory relates to, e.g., reference values or tolerance ranges in which the measurement values may be located. Alternatively or additionally, the data relates to calibration measurements. In this case, the data at least relates to combinations of two measured values with the measure of the stress and, in a configuration, additionally relates to a combination of at least three measured values with the measure of the stress.

In a configuration, the device comprises an energy source. In this case, the energy source is configured in such a way as to supply energy to the evaluation device. In this case, in a configuration, the energy source is a battery or a rechargeable battery. In a further configuration, the energy source allows for “energy harvesting” by converting movements or heat energy of the living being to be examined into electrical energy.

Furthermore, the invention relates to a method for monitoring a state of a living being. In this case, the method includes non-invasively generating a measurement value based on an content of ammonia in the sweat of the living being, generating a measurement value with respect to the respiration or the cardiac activity of the living being and determining from the measurement values—for ammonia and respiration and/or cardiac activity—a scalar measure of a stress of the living being.

In this case, the above-described configurations of the device may also be realized by the method so that the configurations and embodiments accordingly also apply to the method. Thus, repetitions are omitted.

### BRIEF DESCRIPTION OF THE DRAWINGS

Embodiments of the present invention will be detailed subsequently referring to the appended drawings, in which:

FIG. 1 shows a schematic illustration of a living being with an inventive device according to a first variation,

FIG. 2 shows a part of an inventive device according to a second variation as a block diagram,

FIG. 3 shows a schematic section through a first sensor, and

FIG. 4 shows a top view of a first sensor as part of an inventive device.

### DETAILED DESCRIPTION OF THE INVENTION

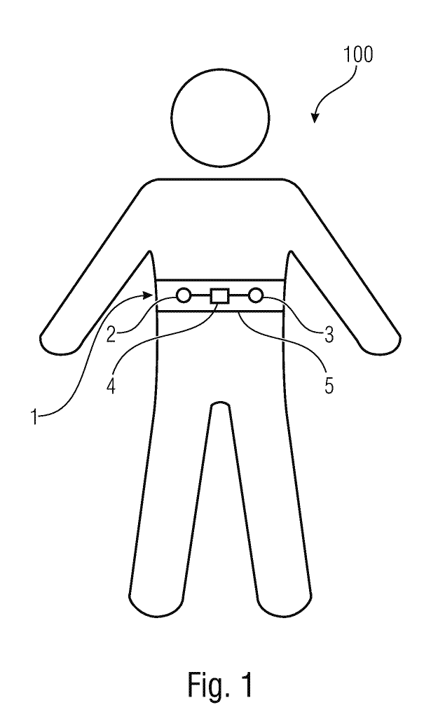

FIG. 1 schematically illustrates an application of the inventive device 1. In this case, the exemplary device 1 is worn as a type of belt at the height of the heart by the living being 100 whose state is to be monitored by the device 1.

The device 1 comprises a first sensor 2 and a second sensor 3 each generating measurement values. The measurement values are directly evaluated on site by the evaluation device 4.

In this case, in the illustrated variation, the first sensor 2 serves for examining the ammonia content in the sweat of the living being 100. The second sensor 3 outputs a measurement value with respect to the cardiac activity. Depending on the configuration, this is a value for the heartbeat, the pulse or, e.g., a blood pressure value or a value regarding the respiration.

The first sensor 2, the second sensor 3 and the evaluation device 4 are located in a carrier device 5 which—as previously mentioned—is configured as a type of belt in this case. In this case, the electronic components 2, 3, 4 are partly located in pockets of the carrier device 5 and are partly inserted directly into the fabric. Alternatively, attachment is provided by means of a hook and loop fastener. In this case, the wiring outlined in the illustration is also located in the fabric. In this case, the two components actually serving for measuring, i.e., the first sensor 2 and second sensor 3, are configured and arranged in the carrier device 5 so that they are located as close as possible to the living being 100.

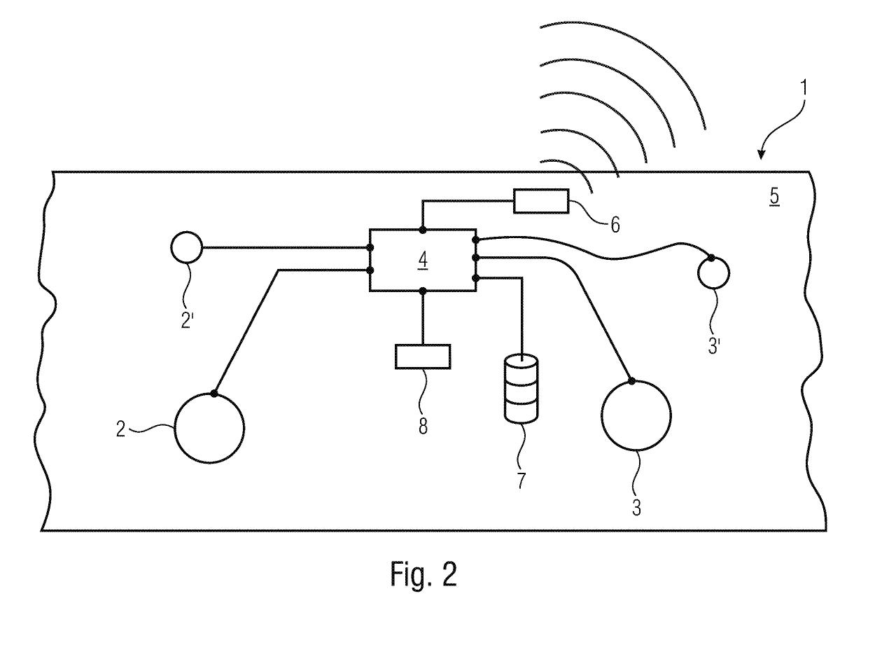

FIG. 2 schematically illustrates parts of a further configuration of the inventive device 1. In this case, the carrier device 5 is only outlined.

Here, two first sensors 2, 2′ are present which each serve for measuring ammonia and which are fixed at different locations. Thus, they allow for a location-specific measurement of the ammonia content.

The first second sensor 3 (here referred to as such) is arranged at a third location and, in the variation illustrated, allows for measuring the cardiac activity, e.g., the pulse via the accordingly provided electrodes. The second second sensor 3′ serves for measuring the respiration. In this case, in a variation, the second second sensor 3′ is configured as a movement sensor and, in a further variation, allows for determining the run time of signals in order to monitor the respiration and determine the respiration rate in a touchless manner.

The measurement values of the two first sensors 2, 2′ as well as of the two second sensors 3, 3′ are supplied in a wired manner to the evaluation device 4 which generates a scalar measure thereof. This measure allows for a statement regarding the current state, in particular regarding the stress of the living being, and is output via an output device 6 by radio. For example, the measure is sent as a measurement result to a display unit (here not illustrated).

In the illustrated configuration, an assessment of the measurement values is carried out by the evaluation device 4 by relating the current measurement values with reference data stored in a data memory 7. Thus, e.g., there is reference data with respect to the ranges in which the ammonia measurement value is to be located in relation to the respiration rate and the pulse. Thus, the measure consists of, e.g., a type of traffic signal, i.e., red, yellow or green, if the reference data includes corresponding value ranges for the measurement values.

The energy supply is carried out via an energy source 8 which is a button cell in the example shown. In this case, the energy source 8 is connected to the evaluation device 4 which, in turn, supplies the energy for the measurement to the first sensors 2, 2′ as well as the second sensors 3, 3′.

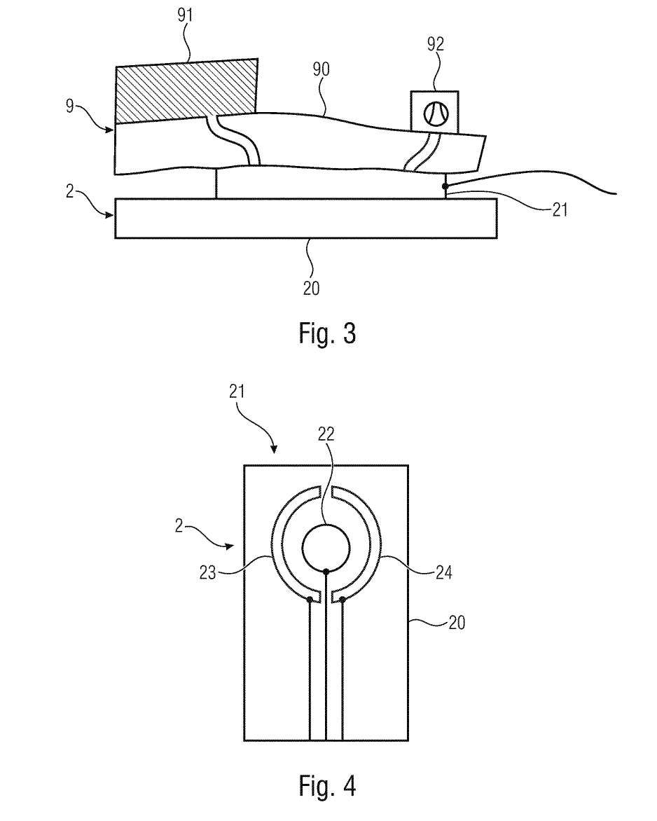

FIG. 3 shows a section through a schematic illustration of a first sensor 2 for measuring an ammonia content. In this case, the first sensor 2 comprises an ion-selective electrode 21 printed on a film 20 (cf. the following FIG. 4), which is connected via an outlined line to the evaluation device (here not shown). In this case, the film 20 is flexible and advantageously also skin-compatible so that it allows for the application under the stress due to the movement of the living being and the immediate skin contact.

In the case illustrated, a transport device 9 which realizes both the transport of the sweat to the first sensor 2 and also the transport away from the first sensor 2 is located on the side of the ion-selective electrode 21 facing away from the film 20.

In this case, in the example shown, the transport device 9 comprises three different components 90, 91, 92:

On the one hand, there is a component 90 provided with channels or capillaries which is arranged directly on the ion-selective electrode 21.

A component 91 comprising an absorbent material is located at an end of a channel facing away from the ion-selective electrode 21. For example, the absorbency is given by the size of pores of the material.

A pumping device 92 which transports away the body fluid sweat after the measurement with the first sensor 2 is located as a third component at an exit of a further channel. Alternatively, an absorbent material which differs from the previously mentioned material 91, e.g., by its pore configuration is also located at this exit.

Here, the first sensor 2 and the transport device 9 are configured with several parts and, in an alternative configuration, are embodied integrally.

FIG. 4 allows for a view of the ion-selective electrode 21 of the first sensor 2, which is applied on a flexible film 20. The concentration of an ion which is a measure of the ammonia content in the sweat of the living being to be examined is determined via the ion-selective electrode 21. In this case, it is advantageously the ammonium ion NH₄⁺.

For this, a circular ion-selective membrane 22 is present which is surrounded by two semi-circular electrodes in the form of a reference electrode 23 and a counter electrode 24. In this case, the ion-selective membrane 22 separates the sweat as measurement medium from the electrode arrangement of the first sensor 2. Accordingly, the membrane 22 is set so that only the desired ions may advantageously pass.

An operating electrode whose electrical contacting is illustrated via a line is also located at the location of the ion-selective membrane 22.

Subsequently, from the measured electrical voltage, the concentration of the ions and e.g., based on calibration data, the ammonia concentration are inferred by the evaluation device.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

### REFERENCES

[1] S. Imani et al., “A wearable chemical-electrophysiological hybrid biosensing system for real-time health and fitness monitoring”, Nat. Commun. 7:11650, doi: 10.1038/ncomms11650, 2016.
[2] W. Gao et al., “Fully integrated wearable sensor arrays for multiplexed in situ perspiration analysis”, Nature, 2016, pp. 509-514.
[3] K. Mitsubayashi et al., “Analysis of metabolites in sweat as a measure of physical condition”, Analytica Chimica Acta, 1994, pp. 27-34.
[4] I. Alvear-Ordenes et al., “Sweat lactate, ammonia, and urea in rugby players”, International Journal of Sports Medicine, 2005, pp. 632-637.
[5] F. Meyer et al., “Effect of age and gender on sweat lactate and ammonia concentrations during exercise in the heat,” Brazilian Journal of Medical and Biological Research, 2007, pp. 135-143.
[6] D. Czarnowski et al., “Plasma ammonia is the principal source of ammonia in sweat,” European Journal of Applied Physiology, 1992, pp. 135-137.
[7] J. M. Lowenstein, “The purine nucleotide cycle revised,” International Journal of Sports Medicine, 1990, pp. 37-46.
[8] H. Schulz et al., “Ammoniak in der Leistungsdiagnostik, Deutsche Zeitschrift für Sportmedizin, 2001, pp. 107-108.
[9] T. Guinovart et al., “Potentiometric sensors using cotton yarns, carbon nanotubes and polymeric membranes”, Analyst, 2013, 5208-5215.

1. A device for monitoring a state of a living being,
comprising several first sensors, a second sensor and an evaluation device,
wherein a first sensor is configured in such a way as to non-invasively generate a measurement value based on a content of ammonia in the sweat of the living being, and a further first sensor is embodied identically, or a further first sensor is configured in such a way as to generate a measurement value for a content of a component in the sweat of the living being other than ammonia,
wherein the first sensors may be positioned at different positions with different muscle groups of the living being,
wherein the first sensors each comprise an ion-selective electrode printed on a film, an ion-selective membrane, an ion-selective material in the form of an ionophore, a reference electrode and a counter electrode,
wherein the second sensor is configured in such a way as to generate a measurement value with respect to the respiration or the cardiac activity of the living being,
wherein the first sensors and the second sensor are arranged in a carrier device wearable at a body of the living being, and
wherein the evaluation device is configured in such a way as to generate a scalar measure of a stress of the living being based on the measurement values of the first sensors and the measurement value of the second sensor.

2. The device according to claim 1,
wherein the first sensors, the second sensor and the evaluation device are arranged in the carrier device.

3. The device according to claim 1,
wherein the device comprises several second sensors, and
wherein one second sensor generates a measurement value with respect to the respiration and another second sensor generates a measurement value with respect to the cardiac activity.

4. The device according to claim 1,
wherein the device comprises at least one transport device, and
wherein the transport device is configured in such a way as to transport the sweat to the first sensor and/or away from the first sensor.

5. The device according to claim 4,
wherein the transport device comprises at least one component provided with at least one channel.

6. The device according to claim 4,
wherein the transport device comprises at least one component comprising an absorbent material.

7. The device according to claim 4,
wherein the transport device comprises at least one pumping device.

8. The device according to claim 1,
wherein the device comprises an output device, and
wherein the output device is configured in such a way as to output the scalar measure generated by the evaluation device.

9. The device according to claim 8,
wherein the output device is configured in such a way as to output the scalar measure by radio.

10. The device according to claim 1,
wherein the device comprises a data memory, and
wherein the evaluation device is configured in such a way as to generate the scalar measure based on the measurement value of the first sensor and the measurement value of the second sensor as well as based on data stored in the data memory.

11. The device according to claim 1,
wherein the device comprises an energy source, and
wherein the energy source is configured in such a way as to supply energy to the evaluation device.
